(19) United States
(12) Patent Application Publication
(10) Pub. No.: US 2024/0197801 A1
(43) Pub. Date: Jun. 20, 2024

(54) Liver Vitality

(71) Applicant: Porche Price, Woodbridge, VA (US)

(72) Inventor: Porche Porche Price, Woodbridge, VA (US)

(21) Appl. No.: 18/083,477

(22) Filed: Dec. 17, 2022

(51) Int. Cl. A61K 36/28; A61K 31/385; A61K 36/9066; A61K 38/54; A61P 1/16

(52) U.S. Cl. CPC A61K 36/28; A61K 31/385; A61K 36/9066; A61K 38/54; A61P 1/16

(57) ABSTRACT

This disclosure relates to administering, manufacturing, and composing oral formulations to treat and prevent fatty liver disease, liver dysfunction, and hepatic impairment. The treatment and prevention may be one or both liver dysfunction-related primary and secondary diseases. The herbal blend may be made of one or more artichokes, dandelion, milk thistle, alpha-lipoic acid, and turmeric. The enzyme blend may be made of one or more isolated enzymes from amylase, glucoamylase, protease 4.5, protease 6, protease 3, neutral protease, peptidase, papain, alpha-galactosidase, acid maltase, cellulase, pectinase, protease inhibitor 6.0, lipase, lactase, bromelain, cellulase, xylanase, beta-glucanase, invertase, hemicellulose, etc., and any combination thereof. The formulation may be in two compartments, the outer and the inner—the outer compartment has the herbal blend and the internal has the enzyme blend. The inner compartment may be gastric acid resistant.

### CROSS-REFERENCE TO RELATED APPLICATIONS: None

This original Nonprovisional Application does not claim benefit to any prior applications.

### STATEMENT REGARDING FEDERALLY SPONSORED RESEARCH OR DEVELOPMENT

Not applicable.

The present disclosure relates to a method of use, administration, manufacture, and composition of oral formulation for treating and preventing fatty liver, liver disease, liver dysfunction-related diseases, and hepatic impairment.

### BACKGROUND

The liver is a major player in the human body's detoxification system and serves several significant functions, including carbohydrate, protein, amino acid, and lipid metabolism. The liver removes chemicals, biochemicals, toxins, and other waste from the blood when the stomach and intestine blood passes through the liver, where it is filtered to regulate and remove chemicals and wastes from the blood. The liver filters and removes several compounds from the body, including hormones such as estrogen and aldosterone, alcohol, and other drugs. Also, for complex molecules such as medications, drugs, and other chemicals, the liver breaks these complex forms into simpler chemical forms to filter and remove from the human body. When the liver has broken down harmful substances, its by-products are excreted into the bile and blood. Later, the bile by-products enter the intestine and leave the body as feces. In addition, the liver is responsible for removing ammonia, a highly toxic chemical that results from protein digestion. The liver converts it into urea by the liver before it can be filtered out by the kidneys and excreted in the urine. Another highly toxic compound is bilirubin, formed by the breakdown of hemoglobin and cleared by the liver; if not cleared, it can accumulate, causing yellowing of the skin and eyes.

Another liver function is to store nutrients for later use by other organs or tissues. The liver can store vitamins A, D, E, K, B₁₂, iron, copper, and glycogen and keeps significant amounts of these vitamins. The liver stores iron from hemoglobin as ferritin, ready to make new red blood cells. Excess blood glucose is converted into the storage form of glycogen, which can later be converted back to glucose for energy, hence regulating blood glucose levels. Because the liver assists in vitamin K absorption, essential for blood clotting, it also regulates blood clotting.

The liver produces bile consisting of bile salts, cholesterol, bilirubin, electrolytes, and water. The bile helps fats break down. In addition, the liver produces cholesterol and other special proteins to help carry fats through the body. Albumin is the most common protein in blood serum produced by the liver. It transports fatty acids and steroid hormones to help maintain the correct pressure and prevent the blood vessels from leaking. The regulation of blood levels of amino acids forms the building blocks of proteins.

The liver regulates blood pressure by angiotensinogen synthesis, which controls blood pressure by modulation of blood vessel contraction and relaxation. The hormone raises blood pressure by narrowing the blood vessels when messaged by producing an enzyme called renin in the kidneys.

The liver is part of the mononuclear phagocyte system. It contains a large number of Kupffer cells that are involved in immune activity. These cells destroy disease-causing agents that might enter the liver through the gut. Moreover, the liver resists infections by making immune factors and removing bacteria from the bloodstream. Therefore, the liver is vital for immune response to prevent infectious diseases.

The formulation of the disclosure herein may improve liver function, thus, assisting in treating or preventing several liver-related diseases.

### BRIEF DESCRIPTION OF THE INVENTION

The formulation provided herein may be used in the treatment and/or prevention of liver dysfunction-related primary and secondary diseases and any combination thereof. The secondary diseases may not be liver-function dysfunction or hepatic impartment, but diseases caused indirectly due to liver disease. The primary diseases may be a fatty liver, abnormal liver enzyme levels, liver disease, liver dysfunction-related diseases, and hepatic impairment. The secondary diseases may be an inability to lose weight (stalled weight loss), weak and slow-growing hair and nail, dull and wrinkled skin, fatigue, type II diabetes, hypercholesteremia, gut dysfunction diseases selected from a group consisting of abnormal food digestion, abnormal mucosal blood flow, abnormal intestinal motility, abnormal gut lining permeability, abnormal nutrient absorption, enhanced allergen absorption, ulcers, abnormal kidney function, an abnormal immune function, autoimmune diseases, inflammation, is abnormal respiration, infections (viral, bacterial, yeast, fungal), alcohol abuse-related liver dysfunction, hormone-balancing, etc., and any combination thereof.

This disclosure pertains to methods of use, administration, manufacture, and composition of an oral formulation made of extracts or powder of a herbal blend, which may be made of one or more artichokes, dandelion, milk thistle, alpha-lipoic acid, and turmeric. The formulation also contains an enzyme blend. The enzyme blend may be made of one or more isolated enzymes selected from a group consisting of amylase, glucoamylase, protease 4.5, protease 6, protease 3, neutral protease, peptidase, papain, alpha-galactosidase, acid maltase, cellulase, pectinase, protease inhibitor 6.0, lipase, lactase, bromelain, cellulase, xylanase, beta-glucanase, invertase, and hemicellulose.

In an embodiment, the extracts may for one or more ingredients may be a tincture, infusions, organic extracts (e.g., alcohol extracts), acid-extracts, hydrophobic liquids such as oils, essential oils, and other edible, non-edible fat extracts, aqueous extracts, non-aqueous extracts, etc., and any combination thereof.

In an embodiment, the powder may be from any plant part, such as a leaf, whole plant, root, seed, stem, bud, flower, flower head, heart, fruit, etc. Mechanical methods may prepare the powder, chemical methods, etc., and any combination thereof.

The composition may be manufactured in one or more physical forms such as but not limited to, emulsion, hydrogel, hydrogel admixture, solutions, patches, pellets, pills, tablets, caplets, capsules, hard-shelled capsules, soft-shelled capsules, drug-embedded capsules, enteric coating, stomach acid resistant capsules, pullulan acid resistant capsule, HPMC acid resistant capsule, delayed-release capsules, oral-deliverables, liquid oral drink, food-additive or supplement, etc., or any combination thereof.

In an embodiment, the enzyme blend may be in a separate physical form from the remaining ingredients and extracts or powder of one or more artichokes, dandelion, milk thistle, alpha-lipoic acid, and turmeric.

In an embodiment, the extracts or powder of one or more artichokes, dandelion, milk thistle, alpha-lipoic acid, and turmeric, etc., may be separated from the enzyme blend such that the enzyme blend may be resistant to gastric acids and/or digestion.

In an embodiment, the oral formulation may be made up of two separate and/or distinct compartments, an outer compartment with herbal blend composition and an inner compartment with an enzyme blend composition.

### DETAILED DESCRIPTION OF THE INVENTION

Examples of embodiments are provided so that this disclosure will be thorough and fully convey the scope to those skilled in the art. Numerous specific details are set forth, such as examples of specific components, devices, and methods, to provide a thorough understanding of the embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components but do not preclude the presence or addition of one or more other features, integers, steps, operations, elements, components, and/or groups thereof. The method steps, processes, and operations described herein are not to be construed as necessarily requiring their performance in the particular order discussed or illustrated unless specifically identified as an order of performance. It is also to be understood that additional or alternative steps may be employed.

The preceding summary, as well as the following detailed description of certain embodiments, will be better understood when read in conjunction with the appended figure of experimental data and results. As used herein, an element or step recited in the singular and proceeded with the word “a” or “an” should be understood as not excluding the plural of said elements or steps unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property. When a definition is provided herein, it supersedes any other meaning or definition.

This disclosure pertains to methods of use, administration, manufacture, and composition of an oral formulation made of extracts or powder of one or more artichokes, dandelion, milk thistle, alpha-lipoic acid, and turmeric, which may be referred to herein as a herbal blend. The formulation also contains an enzyme blend. The enzyme blend may be made of one or more isolated enzymes selected from a group consisting of amylase, glucoamylase, protease 4.5, protease 6, protease 3, neutral protease, peptidase, papain, alpha-galactosidase, acid maltase, cellulase, pectinase, protease inhibitor 6.0, lipase, lactase, bromelain, cellulase, xylanase, beta-glucanase, invertase, hemicellulose, etc., and any combination thereof.

In an embodiment, the formulation may be used by humans for treating or preventing liver disease, improving or enhancing liver function, reducing liver dysfunction-related diseases, reducing fatty liver, enhancing liver function, etc., The formulation provided herein may be used in the treatment and/or prevention of liver disease, improving or enhancing liver function, reducing liver dysfunction-related diseases, reducing fatty liver, enhancing liver function, and any combination thereof.

The formulation may be used in the treatment and/or prevention of liver dysfunction-related diseases, which may be secondary to liver disease, improving or enhancing liver function, reducing liver dysfunction-related diseases, reducing fatty liver, and enhancing liver function. For example, the secondary disease may be reducing obesity, aids in stalled weight loss, maintaining the liver function, reducing fat from the liver, reducing abdominal fat, reduces excess midsection fat, overall weight loss, improving hair strength and density, nail strength, faster-growing nails, skin strength, luminance, anti-wrinkle, reducing fatigue, preventing or reducting the incidence of type II diabetes, improve cholesterol blood levels, aids in digestion, improving gut health and better food digestion like mucosal blood flow, intestinal motility, and the permeability of the gut, improved and proper absorption and helping to reduce the threat of absorption of potential allergens through the gut, aids in filtering toxins from the body, more energy, weight-loss, a better urine regime, reduce ulcers, enhance antioxidant, immune-system booster, aids in respiration, aids in normal inflammation response, treat Candida infections, reducing cancer, improves carbohydrate metabolism, reduces fat absorption, diseases of alcohol abuse, autoimmune diseases, anti-viruses, anti-inflammation, hormone-balancing, etc., and any combination thereof.

The formulation provided herein may be used in the treatment and/or prevention of liver dysfunction-related primary and secondary diseases, fatty liver, and any combination thereof. The secondary diseases may not be liver-function dysfunction or hepatic impartment, but diseases caused indirectly due to liver disease. The primary diseases may be a fatty liver, abnormal liver enzyme levels, liver disease, liver dysfunction-related diseases, and hepatic impairment. The secondary diseases may be an inability to lose weight (stalled weight loss), fatty liver, abnormal liver enzyme levels, weak and slow-growing hair and nail, dull and wrinkled skin, fatigue, type II diabetes, hypercholesteremia, gut dysfunction diseases selected from a group consisting of abnormal food digestion, abnormal mucosal blood flow, abnormal intestinal motility, abnormal gut lining permeability, abnormal nutrient absorption, enhanced allergen absorption, ulcers, abnormal kidney function, an abnormal immune function, autoimmune diseases, inflammation, is abnormal respiration, infections (viral, bacterial, yeast, fungal), alcohol abuse-related liver dysfunction, hormone-balancing, etc., and any combination thereof.

In an embodiment, the formulation may be a medicament for the treatment and/or prevention of liver dysfunction-related diseases, which may be primary and/or secondary to liver diseases, fatty liver, liver dysfunction, and any combination thereof. For example, such secondary may be aids in stalled weight loss, reduces excessive midsection fat, maintaining the liver function, reduces fat from the liver, abdominal fat, overall weight loss, improving hair strength and density, nail strength, faster-growing nails, skin strength, luminance, anti-wrinkle, reducing fatigue, preventing or reducing the incidence of type II diabetes, improve cholesterol blood levels, aids in digestion, improving gut health and better food digestion like mucosal blood flow, intestinal motility, and the permeability of the gut, improved and proper absorption and helping to reduce the threat of absorption of potential allergens through the gut, aids in filtering toxins from the body, more energy, weight-loss, and a better urine regime, reduce ulcers, enhance antioxidant, immune-system booster, aids in respiration, aids in normal inflammation response, treat Candida infections, reducing cancer, improves carbohydrate metabolism, reduces fat absorption, diseases of alcohol abuse, autoimmune diseases, anti-viruses, anti-inflammation, hormone-balancing, etc., and any combination thereof.

In an embodiment, the extracts may for one or more ingredients may be a tincture, infusions, organic extracts (e.g., alcohol extracts), acid-extracts, hydrophobic liquids such as oils, essential oils, and other edible, non-edible fat extracts, aqueous extracts, non-aqueous extracts, etc., and any combination thereof.

In an embodiment, the powder may be from any plant part, such as a leaf, whole plant, root, seed, stem, bud, flower, flower head, heart, fruit, etc. The powder form may be prepared by mechanical methods, chemical methods, etc., and any combination thereof.

In an embodiment, the extracts may be prepared by heating, cooling, pressure, compression, diffusion, chemicals, mechanical disruption, etc., and any combination thereof.

In an embodiment, the extract may be powder, flour, oil, extract, solid, semi-solid, liquid, semi-liquid, resin, diffusable, aerosol, vapor form, or any combination thereof.

In an embodiment, the artichoke or artichoke extract may be from any plant part, such as a leaf, whole plant, root, seed, stem, bud, flower, flower head, heart, fruit, etc. The extract may contain bioactive agent apigenin, luteolin, cynarine, which is a hydroxycinnamic acid derivative, and other antioxidants.

In an embodiment, the artichoke extract may be Cynara cardunculus, or another variety of a species of cultivated thistle as a food belonging to Asteraceae family and/or Cynara genus. For example, Green, big: Vert de Laon, Camus de Bretagne, Castel, Green Globe; Green, medium-size: Verde Palermo, Blanca de Tudela, Argentina, Española, Blanc d′Oran, Sakiz, Bayrampasha; Purple, big: Romanesco, C3; Purple, medium-size: Violet de Provence, Brindisino, Catanese, Niscemese, Violet d′Algerie, Baladi, Ñato, Violetta di Chioggia; Spined: Spinoso Sardo e Ingauno, Criolla; White varieties; Madrigal, Lorca, A-106, Imperial Star; Symphony, Harmony; Purple: Concerto, Opal, Tempo, etc., and any combination thereof.

In an embodiment, the dandelion extract may be from any plant part such as root, leaf, seeds, stem, bud, flower, fruit, etc. The dandelion extract may have one or more phytochemicals such as flavonoids, apigenin, isoquercitrin, quercetin-like, caffeic acid, terpenoids, triterpenes, sesquiterpenes, etc. The dandelion extract might also contain lutein, serine proteinase such as taraxalisin, and prebiotic fiber such as inulin.

In an embodiment, the dandelion may be Taraxacum officinale, or any variety belonging to the dandelion genus in the family Asteraceae. The plants may be a diploid or triploid set of chromosomes. For example, Taraxacum officinale subsp. Ceratophorum (Ledeb.) Schinz ex Thellung, Taraxacum ceratophorum, Taraxacum officinale subsp.officinale, Taraxacum officinale subsp.vulgare (Lam.), subsp. Ceratophorum, Taraxacum officinale L.

In an embodiment, milk thistle extract may be from any plant part such as the leaf, seeds, root, stem, bud, flower, fruit, etc. The milk thistle extract may contain bioactive ingredients such as silymarin.

In an embodiment, milk thistle extract may be Silybum marianum, milk thistle may refer to several plants, such as Lactuca serriola, Silybum marianum, Onopordum acanthium or Cirsium vulgare; various species of Sonchus, including, Sonchus asper, rough milk thistle, Sonchus arvensis, field milk thistle, Sonchus oleraceus, common milk thistle, blessed milk thistle, Marian thistle, Mary thistle, Saint Mary's thistle, Mediterranean milk thistle, variegated thistle Scotch thistle, etc., and any combination thereof.

In an embodiment, the turmeric extract may be from any part of the plant, such as the root, leaf, stem, etc. The turmeric extract may contain phytochemicals and bioactive agents such as curcuminoids, diarylheptanoids, curcumin, dex-ethoxy curcumin, bisdemethoxycurcumin, etc.

In an embodiment, the turmeric extract may have at least one of the 34 essential oils. For example, turmerone, germacrone, atlantone, zingiberene, etc., and any combination thereof.

In an embodiment, the turmeric extract may be Curcuma longa, or any other Curcuma species, and any combination thereof.

In an embodiment, alpha lipoic acid may be isolated from animals such as cattle, pigs, chickens, sheep, lamb, red meat, and organ meat and from plants such as carrots, beets, spinach, broccoli, potatoes, etc., and any combination thereof. It may be isolated from any animal mitochondrial.

In an embodiment, at least one or more of the formulation ingredients or enzyme blend may be in powder, flour, oil, extract, solid, liquid, resin, diffusable, aerosol, vapor form, or any combination thereof.

In an embodiment, the formulation and/or the enzyme blend may be in powder, flour, oil, extract, solid, semi-solid, liquid, semi-liquid, resin, diffusable, aerosol, vapor form, or any combination thereof.

In an embodiment, at least one or more of the formulation ingredients or enzyme blend may be mechanically isolated, chemically isolated, biochemically isolated, isolated by chromatography, solubilization, or a combination thereof, and maybe from a plant, recombinant protein via recombinant DNA/RNA technologies, an animal source, or any combination thereof.

### Enzyme Blend

In an embodiment, the isolated enzyme may be a neutral protease. Neutral proteases may be of four types, depending on the catalytic mechanism and the functional group of the active site: serine protease, aspartic protease, cysteine protease, and metalloprotease. Serine proteases catalyze the hydrolysis of substrate proteins in two steps. During the hydrolysis process, an intermediate dimer of the covalent enzyme-peptide complex is formed, followed by the loss of amino acids and broken peptide chains. The acyl group is then removed, and the peptide bond is hydrolyzed by the nucleophilic attack of the intermediate by water. Most of the serine proteases form a typical triple structure of Ser-His-Asp, and a small amount of serine serves as the catalytic basis.

On the other hand, the activity site of aspartic acid protease is dependent on the aspartic acid residue. The aspartic acid residues in the activity site of most of the pepsin family are located in the Asp-Thr-Gly-Xaa motif, which is located at the bilobate amino and carboxyl ends. Xaa can be replaced by Ser or Thr. The side chain is linked to Asp by hydrogen bonding. The catalytic mechanisms of cysteine protease are similar to those of serine protease. Papain is considered to be the prototype for the catalytic mechanism of cysteine proteases. The difference in the catalytic mechanism is mainly due to the different conformations of active sites: serine proteases contain a Ser-His-Asp catalytic triad, while there is an acid. Cysteine protease: group and an acyl mercaptan intermediate formed during the hydrolysis of cysteine proteases. The metalloproteinase class of enzymes depends on the presence of divalent metal cations and can be inactivated by dialysis or metal chelates. The metal cation is usually Zn²⁺ and may be other metal cations, such as Mg²⁺ and Cu²⁺. The metal ion at the active site of the enzyme can be chelated by a chelating agent, such as EDTA or OP, so that the enzyme loses its partial or full activity. This process is usually reversible, and the enzyme activity can recover by re-adding metal ions.

Neutral proteases are widely present in animals, plants and microorganisms such as fungal, viral, and bacterial. The bacterial neutral proteases may be produced by Bacillus, such as Bacillus subtilis and Bacillus licheniformis. Fungi may product these proteases by fermentation, and the fungi may be Aspergillus oryzae, Rhizopus, and Mucor. The microbial protease may have unparalleled advantages due to its culture conditions, production cost control, and preparation scale.

In an embodiment, the isolated enzyme may be papain. Papain may be produced from the latex of the fruit of the papaya tree. A purification step may be necessary to remove contaminating substances and consists of the solubilization and extraction of the active papain enzyme. The enzyme may be present in papaya or Carica papaya, and mountain papaya or Vasconcellea cundinamarcensis.

In an embodiment, the isolated enzyme is proteases such as 4.5 or 1,4-alpha-D-glucan glucanohydrolase, which functions optimally at a pH of 4.5. This enzyme may be produced by bacterial fermentation using a gram-positive, spore-forming, facultatively anaerobic, rod-shaped bacterium, for example, strains of Bacillus licheniformis. These strains may be isolated and then cultured from the soil, bird feathers, especially chest and back plumage, and most often in ground-dwelling birds, for example, sparrows, and aquatic species, for example, ducks. In addition, these bacterium strains may be used to isolate and manufacture other enzymes of this disclosure.

In an embodiment, the isolated enzyme may be protease 6, which functions best at a pH of 6.8-7.5. This enzyme may be produced by bacterial fermentation using a selected strain of Bacillus licheniformis. Protease inhibitor 6.0 may be from a plant or animal source, for example, saliva, oral tissue, pancreas, or digestive tract of cattle, pigs, chickens, sheep, lamb, etc., and any combination thereof.

In an embodiment, the isolated enzyme may be protease 3 or proteinase 3, a 29-30 kDa serine-protease. It is a weak cationic enzyme, physiologically inhibited by al-antitrypsin, and has a structure with considerable homology to neutrophil elastase. It is expressed only in primates and humans and is found in azurophile (primary) granules of neutrophils, secretory vessels, PMN, monocytes, and plasma membranes.

In an embodiment, the isolate peptidase may be from the stomach, small intestine, digestive tract, and pancreas of cattle, pigs, chickens, sheep, lamb, etc., and any combination thereof. Peptidases are proteases or proteinases responsible for the cleavage of peptide bonds between amino acids via hydrolysis reaction.

In an embodiment, the isolated enzyme may be bromelain, a protein-digesting enzyme mixture derived from the pineapple plant's stem, fruit, and juice.

In an embodiment, the isolated enzyme may be glucoamylase. Amylase breaks down starch into maltose, while glucoamylase breaks down maltose into glucose. It breaks down starch by catalyzing the hydrolysis of 1,4-alpha glucosidic linkage into amylose and amylopectin to yield glucose. It is typically found in the pancreas of mammalians such as cattle, pigs, sheep, lamb, chicken, etc.

In an embodiment, the isolated amylase enzymes may be from saliva, oral tissue, the pancreas, the digestive tract of cattle, pigs, chickens, sheep, lamb, etc., and any combination thereof. Amylase is an enzyme that catalyzes the hydrolysis of starch into simple sugars.

In an embodiment, alpha-galactosidase may be from saliva, oral tissue, pancreas, or digestive tract of cattle, pigs, chickens, sheep, lamb, etc., and any combination thereof. Alpha-galactosidase is a glycoside hydrolase that hydrolysis the terminal alpha-galactosyl moieties from glycolipids and glycoproteins.

In an embodiment, the acid maltase may be from saliva, oral tissue, pancreas digestive tract of cattle, pigs, chickens, sheep, lamb, etc., and any combination thereof. Acid maltase is cellulase and helps to break down glycogen in the lysosome in saliva.

In an embodiment, pectinase may be from plant and/or animal sources, and it can break down pectin from plant cell walls. This may also increase short-chain fatty acids like butyrate for gut bacteria.

In an embodiment, the isolate enzyme may be xylanase such as Endo-1,4-β-xylanase or 4-β-D-xylan xylanohydrolase. This enzyme degrades the linear polysaccharide xylan into xylose, thus breaking down hemicellulose, one of the major components of the plant cell wall. Xylanases are produced by fungi, bacteria, yeast, marine algae, protozoans, snails, crustaceans, insects, seeds, etc., but mammals do not produce xylanases. However, the principal commercial source of xylanases may be filamentous fungi.

In an embodiment, the lactase may be from the pancreas, small intestine, or digestive tract of cattle, pigs, chickens, sheep, lamb, etc., and any combination thereof. Lactase is a lactose hydrolyzing enzyme.

In an embodiment, cellulase may be from plants such as carrots, beets, spinach, broccoli, potatoes, bacteria, fungi, etc., and any combination thereof. Cellulase is a cellulose breakdown enzyme.

In an embodiment, beta-glucanase, which breaks down glycosidic bonds within beta-glucans of carbohydrates, maybe from Beta-glucanase may be from in the cell walls of cereals (e.g., oats, barley), certain types of mushrooms (e.g., reishi, shiitake, maitake), yeasts, seaweed, algae, etc., and any combination thereof.

In an embodiment, invertase may be derived from a beneficial strain of Saccharomyces cerevisiae and then may be purified by itself or as a part of a multi-enzyme formula. Invertase catalyzes the hydrolysis of sucrose into fructose and glucose.

In an embodiment, the hemicellulase may be isolated from microorganisms and/or plant cell walls. It breaks down plant cell walls' hemicellulose, such as pentosans, xylans, galactans, mannans, and glucans. Hemicellulase targets them all, from microorganisms such as microorganisms from the intestinal tract.

In an embodiment, lipase, a fat hydrolyzing enzyme, may be from the pancreas or digestive tract of cattle, pigs, chickens, sheep, lamb, etc., and any combination thereof.

### Other Exemplary Ingredients

In an embodiment, the formulation may have binders, additives, coatings, or fillers such as cellulose, gelatin, agar, rice flour, pectin, orange peel pectin, cellulose derivatives, polyvinyl pyrrolidone, starch, sucrose, mannitol, polyethylene glycol, and liquid glucose, pregelatinized starch, natural dyes derived from beetroot and other plants, plant- or animal-based resins and gums like guar gum, vegetable or animal based magnesium stearate, carmine, shellac, Caprylic acid, tapioca capsules, Pullulan acid resistant capsule, starch capsules, HPMC (hydroxypropyl methylcellulose) capsules, Sucrose, liquid glucose, acacia, tragacanth, gelatin, starch paste, Alginic Acid, cellulose, methylcellulose, ethyl cellulose, Hydroxy Propyl Cellulose, Sodium Carboxy Methyl Cellulose, Polyvinyl Pyrrolidone (PVP), Polyethylene Glycol (PEG), Polyvinyl Alcohols, Polymethacrylates, HPMC acid resistant capsules, Proprietary polymers, Hypromellose, Zein, Sodium Alginate, Shellac, Cellulose acetate trimellitate, Polyvinyl acetate phthalate (PVAP), Cellulose acetate succinate, Cellulose acetate phthalate, Hydroxypropyl methyl cellulose phthalateetc, and any combination thereof.

In an embodiment, the formulation may have preservatives such as silicon dioxide, magnesium stearate, etc., and any combination thereof.

In an embodiment, the formulation may have additional ingredients such as gelatin, etc.

### Method of Use

The oral formulation provided herein may be used in the treatment and/or prevention of liver diseases, fatty liver, liver function, and any combination thereof. The oral formulation may also be used in the treatment and/or prevention of diseases primary and/or secondary to liver diseases, fatty liver, liver function, and any combination thereof. Such secondary diseases may be secondary to liver diseases, fatty liver, liver dysfunction, and any combination thereof. For example, such secondary diseases may be an inability to lose weight (stalled weight loss), fatty liver, abnormal liver enzyme levels, weak and slow-growing hair and nail, dull and wrinkled skin, fatigue, type II diabetes, hypercholesteremia, gut dysfunction diseases selected from a group consisting of abnormal food digestion, abnormal mucosal blood flow, abnormal intestinal motility, abnormal gut lining permeability, abnormal nutrient absorption, enhanced allergen absorption, ulcers, abnormal kidney function, an abnormal immune function, autoimmune diseases, inflammation, is abnormal respiration, infections (viral, bacterial, yeast, fungal), alcohol abuse-related liver dysfunction, hormone-balancing, etc., and any combination thereof.

The composition may be used in addition to complement traditional treatments or cure a variety of liver-related disease and liver diseases, fatty liver, liver function, inability to lose weight (stalled weight loss), fatty liver, abnormal liver enzyme levels, weak and slow-growing hair and nail, dull and wrinkled skin, fatigue, type II diabetes, hypercholesteremia, gut dysfunction diseases selected from a group consisting of abnormal food digestion, abnormal mucosal blood flow, abnormal intestinal motility, abnormal gut lining permeability, abnormal nutrient absorption, enhanced allergen absorption, ulcers, abnormal kidney function, an abnormal immune function, autoimmune diseases, inflammation, is abnormal respiration, infections (viral, bacterial, yeast, fungal), alcohol abuse-related liver dysfunction, hormone-balancing, etc., and any combination thereof.

In an embodiment, the composition may be non-toxic and or without side for the user, wherein users may be humans or non-human vertebrates exposed to the composition.

In an embodiment, an effective amount in one dose of artichokes may be from 112.5 to 600 mg, for example, 225 mg, 100 mg, 200 mg, 225, 250, 300 mg, 350 mg, 400 mg, 450, 500 mg, 550 mg, 600 mg, etc., and any combination thereof.

In an embodiment, an effective amount in one dose of dandelion may be from 75 to 400 mg, for example, 75 mg, 100 mg, 150 mg, 200 mg, 250 mg, 300 mg, 350 mg, 400 mg, etc., and any combination thereof.

In an embodiment, an effective amount in one dose of milk thistle may be from 75 to 400 mg, for example, 75 mg, 100 mg, 150 mg, 200 mg, 250 mg, 300 mg, 350 mg, 400 mg, etc., and any combination thereof.

In an embodiment, an effective amount in one dose of milk thistle may be up to 90% Silybum, for example, 50%, 60%, 70%, 75%, 80%, 85%, 90%, etc., and any combination thereof.

In an embodiment, an effective amount in one dose of alpha-lipoic acid may be from 50 to 200 mg, for example, 50 mg, 100 mg, 150 mg, 200 mg, etc., and any combination thereof.

In an embodiment, the alpha lipoic acid may be 200 to 1,800 mg daily, such as 300, 400, 500, 600, 700, 800, 900, 1000, 1100, 1200, 1300, 1400, 1500, 1600, 1700, or 1800 mg daily. In an embodiment, the alpha lipoic acid may be taken 1, 2, 3, 4, 5, 6, 7, or 8 times daily.

In an embodiment, an effective amount in one dose of turmeric may be from 75 to 600 mg, for example, 75 mg, 100 mg, 150 mg, 200 mg, 250 mg, 300 mg, 350 mg, 400 mg, 450 mg, 500 mg, 550 mg, 600 mg, etc., and any combination thereof.

In an embodiment, an effective amount of turmeric may be standardized up to 99% curcuminoids, for example, 70%, 75%, 80%, 85%, 90%, 95%, 96%, 97%, 98%, etc., and any combination thereof.

In an embodiment, an effective amount in one dose of amylase may be up to 6,000 DU, for example, 10, 20, 30, 40, 50, 60, 70, 80, 90, 100, 150, 200, 300, 400, 500, 1000, 1500, 2000, 2500, 3000, 3200, 3255, 3500, 4000, 4500, 5000, 5500, 6000 DU, etc., and any combination thereof.

In an embodiment, an effective amount in one dose of protease 4.5 may be up to 16,000 HUT, for example, 10, 20, 30, 40, 50, 60, 70, 80, 90, 100, 110, 120, 130, 140, 150, 160, 170, 180, 190, 200, 300, 400, 500, 1000, 1500, 2000, 2500, 3000, 3200, 3255, 3500, 4000, 4500, 5000, 5500, 6000, 6500, 70000, 7500, 8000, 8200, 8500, 8600, 9000, 9500, 10000, 11000, 12000, 12500, 13000, 13500, 14000, 14500, 15000, 15500, 16000 HUT, etc., and any combination thereof.

In an embodiment, an effective amount in one dose of protease 6 may be up to 1600 HUT, for example, 10, 20, 30, 40, 50, 60, 70, 80, 90, 100, 110, 120, 130, 140, 150, 160, 170, 180, 190, 200, 300, 400, 500, 550, 600, 650, 7000, 750, 800, 820, 850, 860, 900, 950, 1000, 1050, 1,085, 1100, 1200, 1250, 1300, 1350, 1400, 1450, 1500, 1550, 1600 HUT, etc., and any combination thereof.

In an embodiment, an effective amount in one dose of protease 3 may be up to 10 SAPU, for example, 0.04, 0.05. 0.6, 0.07, 0.08, 0.09, 0.1, 0.2, 0.3, 0.4, 0.5, 1, 1.5, 2, 2.5, 3, 3.5, 4, 4.5, 4.55, 5, 5.5, 6, 6.5, 7, 7.5, 8, 8.5, 9, 9.5, 10 SAPU, etc., and any combination thereof.

In an embodiment, an effective amount in one dose of neutral protease may be up to 2600 PC, for example, 1, 1.5, 2, 2.5, 3, 3.5, 4, 4.5, 4.55, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 35, 40, 45, 50, 100, 200, 300, 400, 500, 550, 600, 650, 7000, 750, 800, 820, 850, 860, 900, 950, 1000, 1050, 1,085, 1100, 1200, 1250, 1300, 1350, 1400, 1450, 1500, 1550, 1600 1,6277, 1650, 1700, 1750, 1800, 1850, 1900, 1950, 2000, 2050, 2100, 2150, 2200, 2250, 2300, 2350, 2400, 2450, 2500, 2550 PC, etc., and any combination thereof.

In an embodiment, an effective amount in one dose of acid maltase may be up to 10 U, for example, 0.04, 0.05. 0.6, 0.07, 0.08, 0.09, 0.1, 0.2, 0.3, 0.4, 0.5, 1, 1.5, 2, 2.5, 3, 3.5, 4, 4.5, 4.55, 5, 5.5, 6, 6.5, 7, 7.5, 8, 8.5, 9, 9.5, 10 U, etc., and any combination thereof.

In an embodiment, an effective amount in one dose of bromelain may be up to 300 FCC PU, for example, 0.04, 0.05. 0.6, 0.07, 0.08, 0.09, 0.1, 0.2, 0.3, 0.4, 0.5, 1, 1.5, 2, 2.5, 3, 3.5, 4, 4.5, 4.55, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 130, 140, 150, 160, 162, 163, 264, 165, 166, 170, 180, 190, 200, 210, 220, 230, 240, 250, 260, 270, 280, 290, 300 FCC PU, etc., and any combination thereof.

In an embodiment, an effective amount in one dose of papain may be up to 300 USP, for example, 0.04, 0.05. 0.6, 0.07, 0.08, 0.09, 0.1, 0.2, 0.3, 0.4, 0.5, 1, 1.5, 2, 2.5, 3, 3.5, 4, 4.5, 4.55, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 130, 140, 150, 151, 152, 153, 154, 155, 156, 157, 158, 159, 160, 162, 163, 164, 165, 166, 170, 180, 190, 200, 210, 220, 230, 240, 250, 260, 270, 280, 290, 300 USP, etc., and any combination thereof.

In an embodiment, an effective amount in one dose of glucoamylase may be up to 10 AGU, for example, 0.04, 0.05. 0.6, 0.07, 0.08, 0.09, 0.1, 0.2, 0.3, 0.4, 0.5, 1, 1.5, 2, 2.5, 3, 3.5, 4, 4.5, 4.5, 5, 5.1, 5.2, 5.3, 5.4, 5.5, 5.6, 5.7, 5.8, 5.9, 6, 6.5, 7, 7.5, 8, 8.5, 9, 9.5, 10 AGU, etc., and any combination thereof.

In an embodiment, an effective amount in one dose of peptidase may be up to 2550 HUT, for example, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 130, 140, 150, 151, 152, 153, 154, 155, 156, 157, 158, 159, 160, 162, 163, 164, 165, 166, 170, 180, 190, 200, 210, 220, 230, 240, 250, 260, 270, 280, 290, 300, 350, 400, 450, 500, 550, 600, 650, 7000, 750, 800, 820, 850, 860, 900, 950, 1000, 1050, 1,085, 1100, 1200, 1250, 1300, 1350, 1400, 1450, 1500, 1550, 1600 1,6277, 1650, 1700, 1750, 1800, 1845, 1846, 1849, 1850, 1,851, 1582, 1883, 1884, 1885, 1890, 1895, 1900, 1950, 2000, 2050, 2100, 2150, 2200, 2250, 2300, 2350, 2400, 2450, 2500, 2550 HUT, etc., and any combination thereof.

In an embodiment, an effective amount in one dose of lactase may be up to 700 ALU, for example, 0.5, 1, 1.5, 2, 2.5, 3, 3.5, 4, 4.5, 4.5, 5, 5.1, 5.2, 5.3, 5.4, 5.5, 5.6, 5.7, 5.8, 5.9, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 130, 140, 150, 151, 152, 153, 154, 155, 156, 157, 158, 159, 160, 162, 163, 164, 165, 166, 170, 180, 190, 200, 210, 220, 230, 240, 250, 260, 270, 280, 290, 300, 310, 320, 330, 340, 344, 346, 347, 348, 349, 350, 351, 352, 353, 354, 355, 356, 357, 358, 359, 360, 362, 363, 364, 365, 366, 370, 380, 390, 400, 410, 420, 430, 440, 450, 460, 470, 480, 490, 370, 380, 390, 400, 500, 510, 520, 530, 540, 550, 560, 570, 580, 590, 600, 650, 660, 670, 680, 690, 700 ALU, etc., and any combination thereof.

In an embodiment, an effective amount in one dose of alpha-galactosidase may be up to 300 GALU, for example, 0.3, 0.4, 0.5, 1, 1.5, 2, 2.5, 3, 3.5, 4, 4.5, 4.5, 5, 5.1, 5.2, 5.3, 5.4, 5.5, 5.6, 5.7, 5.8, 5.9, 6, 6.5, 7, 7.5, 8, 8.5, 9, 9.5, 10, 15, 20, 25, 30, 35, 40, 41, 42, 43, 44, 45, 46, 47, 48, 49, 50, 55, 60, 65, 70, 75, 80, 85, 90, 95, 100, 110, 120, 130, 140, 150, 160, 162, 163, 264, 165, 166, 170, 180, 190, 200, 210, 220, 230, 240, 250, 260, 270, 280, 290, 300 GALU, etc., and any combination thereof.

In an embodiment, an effective amount in one dose of lipase may be 600 FIP, for example, 0.5, 1, 1.5, 2, 2.5, 3, 3.5, 4, 4.5, 4.5, 5, 5.1, 5.2, 5.3, 5.4, 5.5, 5.6, 5.7, 5.8, 5.9, 6, 6.5, 7, 7.5, 8, 8.5, 9, 9.5, 10, 15, 20, 25, 30, 35, 40, 41, 42, 43, 44, 45, 46, 47, 48, 49, 50, 55, 60, 65, 70, 75, 80, 85, 90, 95, 100, 110, 120, 130, 140, 150, 160, 162, 163, 264, 165, 166, 170, 180, 190, 200, 210, 220, 230, 240, 250, 260, 270, 280, 290, 300, 310, 320, 330, 340, 350, 360, 370, 380, 390, 400, 410, 420, 430, 440, 450, 451, 452, 453, 454, 455, 456, 457, 458, 459, 460, 470, 480, 490, 500, 510, 520, 530, 540, 550, 560, 570, 580, 590, 600, FIP, etc., and any combination thereof.

In an embodiment, an effective amount in one dose of pectinase may be up to 10 Endo PG, for example, 0.01, 0.02, 0.03, 0.04, 0.05. 0.6, 0.07, 0.08, 0.09, 0.1, 0.2, 0.3, 0.4, 0.5, 1, 1.5, 2, 2.5, 3, 3.5, 4, 4.5, 4.5, 5, 5.1, 5.2, 5.3, 5.4, 5.5, 5.6, 5.7, 5.8, 5.9, 6, 6.5, 7, 7.5, 8, 8.5, 9, 9.5, 10 Endo PG, etc., and any combination thereof.

In an embodiment, an effective amount in one dose of invertase may be up to 400 SU, for example, 0.04, 0.05. 0.6, 0.07, 0.08, 0.09, 0.1, 0.2, 0.3, 0.4, 0.5, 1, 1.2, 1.3, 1.4, 1.5, 1.6, 1.7, 1.8, 1.9, 2, 2.5, 3, 3.5, 4, 4.5, 4.5, 5, 5.1, 5.2, 5.3, 5.4, 5.5, 5.6, 5.7, 5.8, 5.9, 6, 6.5, 7, 7.5, 8, 8.5, 9, 9.5, 10 5, 10, 15, 20, 25, 30, 35, 40, 41, 42, 43, 44, 45, 46, 47, 48, 49, 50, 55, 60, 65, 70, 75, 80, 85, 90, 95, 100, 110, 120, 130, 140, 150, 160, 162, 163, 264, 165, 166, 170, 180, 181, 182, 183, 184, 185, 186, 187, 188, 189, 190, 200, 210, 220, 230, 240, 250, 260, 270, 280, 290, 300, 310, 320, 330, 340, 350, 360, 370, 380, 390, 400 SU, etc., and any combination thereof.

In an embodiment, an effective amount in one cellulase dose may be up to 300 CU, for example, 0.04, 0.05. 0.6, 0.07, 0.08, 0.09, 0.1, 0.2, 0.3, 0.4, 0.5, 1, 1.1, 1.2, 1.3, 1.4, 1.5, 1.6, 1.7, 1.8, 1.9, 2, 2.5, 3, 3.5, 4, 4.5, 5, 5.1, 5.2, 5.3, 5.4, 5.5, 5.6, 5.7, 5.8, 5.9, 6, 6.5, 7, 7.5, 8, 8.5, 9, 9.5, 10 5, 10, 15, 20, 25, 30, 35, 40, 41, 42, 43, 44, 45, 46, 47, 48, 49, 50, 55, 60, 65, 70, 71, 72, 73, 74, 75, 76, 77, 78, 79, 80, 85, 90, 95, 100, 110, 120, 130, 140, 150, 160, 162, 163, 264, 165, 166, 170, 180, 190, 200, 210, 220, 230, 240, 250, 260, 270, 280, 290, 300 CU, etc., and any combination thereof.

In an embodiment, an effective amount in one xylanase dose may be 300 XU, for example, 0.04, 0.05. 0.6, 0.07, 0.08, 0.09, 0.1, 0.2, 0.3, 0.4, 0.5, 1, 1.1, 1.2, 1.3, 1.4, 1.5, 1.6, 1.7, 1.8, 1.9, 2, 2.5, 3, 3.5, 4, 4.5, 4.5, 5, 5.1, 5.2, 5.3, 5.4, 5.5, 5.6, 5.7, 5.8, 5.9, 6, 6.5, 7, 7.5, 8, 8.5, 9, 9.5, 10, 15, 20, 25, 30, 35, 40, 41, 42, 43, 44, 45, 46, 47, 48, 49, 50, 55, 60, 65, 70, 75, 80, 85, 90, 95, 100, 105, 106, 107, 108, 109, 110, 120, 130, 140, 150, 160, 162, 163, 264, 165, 166, 170, 180, 190, 200, 210, 220, 230, 240, 250, 260, 270, 280, 290, 300 XU, etc., and any combination thereof.

In an embodiment, an effective amount of beta-glucanase may be 100 mg to 500 mg daily, for example, 150, 200, 250, 300, 350, 400, 450 mg, etc., and any combination thereof.

In an embodiment, an effective amount of hemicellulose may be from 20-60 mg per day, for example, 25, 30, 35, 40, 45, 50, 55 mg daily, etc., and any combination thereof.

In an embodiment, an effective amount in one dose of the enzyme blend may be from 25 mg to 200 mg, for example, 50 mg, 75 mg, 10 mg, 125 mg, 150 mg, 200 mg, etc., and any combination thereof.

In one embodiment, the composition with the herbal composition may be given at a dose of 35-1500 mg per dose, for example, 30, 34, 35, 36, 37, 38, 39, 40, 41, 42, 43, 44, 45, 46, 47, 48, 49, 50, 51, 52, 53, 54, 55, 56, 60, 65, 70, 75, 80, 85, 90, 95, 100, 150, 200, 250, 300, 350, 400, 450, 500, 550, 600, 650, 675, 700, 750, 800, 850, 900, 950, 1000, 1100, 1200, 1300, 1400, 1500 mg or any combination for humans.

In an embodiment, the enzyme blend composition may be be given at a dose of 25-150 mg per dose, for example, 30, 34, 35, 36, 37, 38, 39, 40, 41, 42, 43, 44, 45, 46, 47, 48, 49, 50, 51, 52, 53, 54, 55, 56, 60, 65, 70, 75, 80, 85, 90, 95, 100 mg, 110 mg, 115, 120, 125, 130, 135, 140, 145, 150 mg or any combination for humans.

In an embodiment, total active ingredients (the herbs plus the enzyme blend) composition may be given at a dose of 35-2500 mg per dose, for example, 30, 34, 35, 36, 37, 38, 39, 40, 41, 42, 43, 44, 45, 46, 47, 48, 49, 50, 51, 52, 53, 54, 55, 56, 60, 65, 70, 75, 80, 85, 90, 95, 100, 100, 150, 200, 250, 300, 350, 400, 450, 500, 550, 600, 650, 675, 700, 750, 800, 825, 850, 900, 950, 1000, 1100, 1200, 1300, 1400, 1500, 1600, 1700, 1800, 1900, 2000, 2100, 2200, 2300, 2400, 2500 mg or any combination thereof for humans.

In an embodiment, a user may consume several forms and a number of doses ranging from one to a few to many spanning over a few minutes, hours, days, weeks, months, or years. For example, the user may consume this composition in 1, 2, 3, 4, 5, 6, 7, 8, 9, 10, 11, 12, 15, 16, 17, 18, 19, 20, 21, 22, 23, 24, 25, 26, 27, 28, 29, 30-35, 35-40, 40-45, 45-50, more doses, or any combination thereof, and spanning over a few minutes, hours, days, for example, 1, 2, 3, 4, 5, 6, 7, 8, 9, 10, 11, 12, 13, 14, 15, 16, 17, 18, 19, 20-25, 25-30, 30-35, 35-40, 40-45, 45-50, 50-55, 55-60, or more days or any combination thereof; 1, 2, 3, 4, 5, 6, 7, 8, 9, 10, 11, 12, 13, 14, 15, 16, 17, 18, 19, 20, 21, 22, 23, or more weeks or any combination thereof; 1, 2, 3, 4, 5, 6, 7, 8, 9, 10, 11, 12, 13, 14, 15, 16, 17, 18, 19, 20, or more months or any combination thereof; or upto 1, 2, 3, 4, 5, more years or any combination thereof.

In an embodiment, the formulation may be used for at least 1, 2, 3, 4, 5, 6, 7, 8, 9, 10, 11, 12, 13, 14, 15, 16, 17, 18, 19, 20, 21, 22, 23, or 24 weeks before any significant observable impact, such as alternation in biochemical biomarkers, appearance, improvement in symptoms, improvement in biomarkers or markers of a disease, etc., and any combination thereof.

### Exemplary Delivery Systems

The composition may be manufactured in several physical forms such as but not limited to, emulsion, hydrogel, hydrogel admixture, solutions, patches, pellets, pills, tablets, caplets, capsules, hard-shelled capsules, soft-shelled capsules, drug-embedded capsules, enteric coating, stomach acid resistant capsules, Pullulan acid resistant capsule, HPMC acid resistant capsule, delayed-release capsules, oral-deliverables, liquid oral drink, food-additive or supplement, etc., or any combination thereof.

In an embodiment, at least one or more formulation ingredients or enzyme blends may be in powder, flour, oil, extract, solid, liquid, resin, diffusable, aerosol, vapor form, or any combination thereof.

In an embodiment, the formulation and/or the enzyme blend may be in powder, flour, oil, extract, solid, semi-solid, liquid, semi-liquid, resin, diffusable, aerosol, vapor form, or any combination thereof.

In an embodiment, at least one or more of the formulation ingredients or enzyme blend may be mechanically isolated, chemically isolated, biochemically isolated, isolated by chromatography, solubilization, or a combination thereof, and maybe from a plant, recombinant protein via recombinant DNA/RNA technologies, an animal source, or any combination thereof.

In an embodiment, the capsule volume may be up to 1.5 ml, for example, 0.37, 0.5, 0.68, 0.93, 1.37 ml, etc., and any combination thereof.

In an embodiment, the capsule length may be up to 30 mm, for example, 17.5, 19.2, 21.3, 23.6, 26.14 mm, etc., and any combination thereof.

In an embodiment, the capsule may hold the entire blend weight of 200 mg to 1700 mg, for example, 442.5, 885, 822, 558, 408, 300, 222, 1096, 744, 544, 400, 296, 1370, 930, 680, 500, 370, 1644, 1116, 816, 600, 444 mg, etc., and any combination thereof.

### Exemplary Symptoms and Disease Biomarkers

In an embodiment, the formulation may reduce fatty liver fat by 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, the formulation may reduce midsection fat by 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, the formulation may reduce overall fat or obesity by 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, the formulation may improve hair strength and/or density by 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, the formulation may improve nail strength and/or speed of growth by 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, improve skin strength, luminance, anti-wrinkle, 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, reducing fatigue, 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, preventing or reducing the incidence of type II diabetes, 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, improve cholesterol blood levels, 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, it aids in digestion, improving gut health 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, improve urine regime, 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, reduce ulcers, 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, immune-system booster, 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, it improves carbohydrate metabolism, 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

In an embodiment, it improves Basal Metabolic Index (BMI), 0.5%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, etc., and any combination thereof.

The liver function-determining enzyme may be Alanine transaminase (ALT), Aspartate transaminase (AST), Alkaline phosphatase (ALP), Gamma-glutamyltransferase (GGT), L-lactate dehydrogenase (LD). Increased blood levels of these enzymes may suggest a reduced or impaired liver function. Other liver function determinants are prothrombin time (PT), albumin and total protein, and bilirubin. An increase in PT and bilirubin blood levels suggests impaired or reduced liver function, whereas increased albumin indicates normal or improved liver function.

In an embodiment, the oral composition may improve the Alanine transaminase (ALT) blood levels to 7 to 56 U/L (units per liter).

In an embodiment, the oral composition may improve the Alkaline phosphatase (ALP) blood levels to 44 to 147 U/L (units per liter).

In an embodiment, the oral composition may improve the Aspartate transaminase (AST) blood test is 8 to 33 U/L (units per liter).

In an embodiment, the oral composition may improve the Gamma-glutamyltransferase ranges to 15-85 IU/L for men and 5-55 IU/L for women.

In an embodiment, the oral composition may improve the L-lactate dehydrogenase (LD) ranges from 50 to 150 U/L.

In an embodiment, the oral composition may improve the prothrombin time determined in International Sensitivity Index (ISI) and may be between 0.94 ISI and 1.4 ISI for more sensitive and 2.0-3.0 ISI for less sensitive thromboplastins.

In an embodiment, the oral composition may improve the normal albumin range of 3.5 to 5.5 g/dL or 35-55 g/liter, which may be albumin composed of 50%-60% of blood plasma proteins.

In an embodiment, the oral composition may improve the bilirubin test results expressed as direct, indirect, or total bilirubin. For example, total bilirubin may be a combination of direct and indirect bilirubin; for example, normal results for a total bilirubin test may be 1.2 milligrams per deciliter (mg/dL) for adults and usually 1 mg/dL for those under 18, and normal results for direct bilirubin may generally 0.3 mg/dL.

Diabetes may be determined by measuring the A1C blood levels, fasting blood sugar levels, glucose tolerance, and random blood sugar levels. The A1C blood level measures the average blood sugar level over the past 2 or 3 months. An A1C below 5.7% is normal, between 5.7 and 6.4% indicates prediabetes, and 6.5% or higher indicates diabetes. A fasting blood sugar test measures blood sugar after an overnight fast. A fasting blood sugar level of 99 mg/dL or lower is normal, 100 to 125 mg/dL indicates prediabetes, and 126 mg/dL or higher indicates diabetes. The glucose tolerance test measures blood sugar before and after a patient drinks a liquid that contains glucose. This is done after overnight fasting, and then blood sugar levels are measured before the drink, after 1 hour, 2 hours, and 3 hours of drinking the glucose drink. At 2 hours, a blood sugar level of 140 mg/dL or lower is considered normal, 140 to 199 mg/dL indicates prediabetes, and 200 mg/dL or higher indicates diabetes. Finally, a random blood sugar test measures a patient's blood sugar at any time without needing the previous fasting. A blood sugar level of 200 mg/dL or higher indicates diabetes.

In an embodiment, the oral formulation composition may improve the A1C blood levels to the normal range of below 5.7%, 5.8, 6.4%, 6.5, etc., and any combination thereof.

In an embodiment, the oral formulation composition may improve the fasting blood glucose levels to the normal range of 70-100 mg/dl.

Detecting inflammation in a body may be determined by measuring hemoglobin A1c, C-reactive protein, serum ferritin, erythrocyte sedimentation rate, and platelet blood levels.

In an embodiment, the oral formulation composition may improve the C-reactive protein blood levels to a normal value of less than 3.0 mg/L, less than 10 mg/L, 100 mg/L, 500 mg/L, etc., and any combination thereof.

In an embodiment, the oral formulation composition may improve the serum ferritin blood levels to the normal range for a female of 14.7 to 205.1 nanograms per milliliter (ng/ml), and for a male of 30.3 to 565.7 ng/mL.

In an embodiment, the oral formulation composition may improve the erythrocyte sedimentation rates to the normal range for a male of 0 up to 15 mm/hour, for a female of 0 up to 20 mm/hour, for 50 years or older male of 0 to 20 mm/hour, and 50 years or older female of 0 to 30 mm/hour, a child of up to 10 mm/hour, and a newborn=0 to 2 mm/hour.

In an embodiment, the oral formulation composition may improve the platelet blood levels to the normal range of 150,000 to 400,000 platelets per microliter (mcL) or 150 to 400×109/L.

In an embodiment, the oral formulation composition may improve and increase physical energy by up to 100%, for example, 20, 25, 30, 35, 40, 45, 50, 55, 60, 65, 70, 75, 80, 85, 90, 95, and 100%.

In an embodiment, the oral formulation composition may improve and increase mental clarity by up to 100%, for example, 20, 25, 30, 35, 40, 45, 50, 55, 60, 65, 70, 75, 80, 85, 90, 95, and 100%.

In an embodiment, the oral formulation composition may improve and increase physical energy and/or mental within one year, for example, within 1, 2, 3, 4, 5, 6, 7, 8, 9, or 10 days, 1, 2, 3, 4, 5, 6, 7, or 8 weeks, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12 months.

In an embodiment, the oral formulation composition may reduce waist inches by up to 10 inches, for example, 1, 1.5, 2, 2.5, 3, 3.5, 4, 4.5, 5, 5.5, 6, 6.5, 7, 7.5, 8, 8.5, 9, 9.5, or 10 inches.

In an embodiment, the oral formulation composition may reduce weight by up to 200 pounds, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 120, 130, 140, 150, 160, 170, 180, 190, or 200 pounds.

In an embodiment, the oral formulation composition may reduce waist inches and/or overall weight within one year, for example, within 1, 2, 3, 4, 5, 6, 7, 8, 9, or 10 days, 1, 2, 3, 4, 5, 6, 7, or 8 weeks, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12 months.

In an embodiment, the oral formulation composition may assist in weight and/or dimension (in inches) maintenance. The maintenance may be for as long as the person takes the composition regularly for 1, 2, 3, 4, 5, 6, 7, 8, 9, or 10 days, 1, 2, 3, 4, 5, 6, 7, or 8 weeks, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12 months.

In an embodiment, the Amylase may be from 15,000 to 85,000 Internation Units (IU) daily, for example, 16,000, 17,000, 18000, 19000, 20,000, 25,000, 30,000, 40,000, 45,000, 50,000, 55,000, 60,000, 65,000, 70,000, 75,000, 76,000, 77,000, 78,000, 79,000, 80,000, 81,000, 82,000, 85,000, etc., and any combination thereof.

In an embodiment, the alpha-galactosidase may be 300 to 1,500 GalU daily, for example, 350, 400, 450, 500, 550, 600, 650, 700, 750, 800, 850, 900, 950, 1000, 1050, 1100, 1150, 1200, 1300, 1400, 1500, etc., and any combination thereof.

In an embodiment, cellulase may be from 500 mg to 19 gms, for example, 600 mg, 750 mg, 1 gm, 1.5 gm, 2 gm, 3, 4, 5, 6, 7, 8, 9, 10, 11, 1, 13, 14, 15, 16, 17, 18, 19 mg, etc., and any combination thereof.

In an embodiment, an effective amount of protease inhibitor 6.0 may be from 100 to 400 mg daily, for example, 100, 150, 200, 250, 300, 350, 400 mg per day, etc., and any combination thereof.

In an embodiment, an effective amount of lactase may be from 6,000 to 9,000 IU daily, for example, 6500, 7000, 7500, 8500, 9000 IU daily, etc., and any combination thereof.

In an embodiment, an effective amount of beta-glucanase may be 100 mg to 500 mg daily, for example, 150, 200, 250, 300, 350, 400, 450 mg, etc., and any combination thereof.

In an embodiment, an effective amount of hemicellulose may be from 20-60 mg per day, for example, 25, 30, 35, 40, 45, 50, 55 mg daily, etc., and any combination thereof.

### Example

Composition A

| INGREDIENT | AMOUNT PER DOSE |
|---|---|
| Artichokes | 110-250 mg |
| Dandelion | 125-160 mg |
| milk thistle | 125-160 mg |
| alpha-lipoic acid | 125-160 mg |
| Turmeric | 75-125 mg |
| Enzyme Blend | 20-60 mg |

Composition B

| ENZYMES | ENZYME ACTIVITY PER DOSE |
|---|---|
| Amylase | 30-1,500 DU |
| Glucoamylase | 0.055-3 AGU |
| protease 4.5 | 85-4,250 HUT |
| protease 6 | 10-500 HUT |
| protease 3 | 0.43-22 SAPU |
| neutral protease | 16-800 PC |
| Peptidase | 10-500 HUT |
| Papain | 1.5-75 USP |
| alpha-galactosidase | 0.44-22 GAL |
| acid maltase | 0.045-2.5 U |
| Cellulase | 75-3750 CU |
| Pectinase | 0.03-15 HUT |
| Lipase | 4.55-230FIP |
| Lactase | 3.5-15,250 ALU |
| Bromelain | 1.6-80 FCC PU |
| Cellulase | 0.76-40 CU |
| Xylanase | 1.08-50 XU |
| Invertase | 1.9-100 SU |

A patient with hepatic dysfunction may be administered the above composition A, B, or both orally 2 or 3 times daily for up to 12 weeks, for example, 2, 3, 4, 5, 6, 7, 8, 9, 10, 11, 12 weeks. The oral formulation may be made up of two separate and/or distinct compartments, an outer compartment with composition A (herbal blend), which has an inner compartment with composition B (enzyme blend). The inner compartment may be made of a hard-shelled capsule, soft-shelled capsules, drug-embedded capsules, enteric coating, stomach acid-resistant capsules, Pullulan acid-resistant capsules, HPMC acid-resistant capsules, delayed-release capsules, etc., or any combination thereof. The outer compartment may be configured to release its content in the stomach so the stomach's acid may entirely or partially digest it. However, the inner compartment with the enzyme blend may be resistant to stomach (gastric acid) acid degradation, so the enzyme blend may be released in the small, large intestine, or both, where the enzymes may perform their function. The enzymes may aid food digestion and/or enhance gut microbiome growth.

Examples: Below are examples of the use and resultant effects of the composition of the disclosure herein. It is understood that these examples are illustrative purposes and are not limiting the use or the resultant effects. Other examples with similar examples not disclosed herein are possible.

Person 1 took two capsules per day, one dose daily, and experienced effects on the first day, increased physical energy by 50% and mental clarity by 30%.

Person 2 took one dose per day for two weeks and observed effects within one week, increased physical energy by 80% and maintained their current weight with no weight gain.

Person 3 took one dose daily for three months, lost 4 inches off the waist, and lost 40 pounds at the end of three months.

Person 4 took one dose daily for three months, felt the effects within three days, and physical energy increased by 100%, which was maintained for three months.

Person 5 took half a dose daily and felt the effects on the first day, increasing physical energy by 50%.

It is to be understood that the above description is intended to be illustrative and not restrictive. For example, the above-described embodiments (and/or aspects thereof) may be used in combination with each other. In addition, many modifications may be made to adapt a particular situation, method, system device or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claim.

We claim:

1. A method of treating and/or preventing liver dysfunction-related disease by administering an oral formulation comprising of a herbal blend and an enzyme blend, wherein the herbal blend comprises at least one of artichokes, dandelion, milk thistle, alpha-lipoic acid, and turmeric; and wherein the enzyme blend comprises of at least one isolated enzyme of amylase, glucoamylase, protease 4.5, protease 6, protease 3, neutral protease, peptidase, papain, alpha-galactosidase, acid maltase, cellulase, pectinase, protease inhibitor 6.0, lipase, lactase, bromelain, cellulase, xylanase, beta-glucanase, invertase, and hemicellulose.

2. The method of claim 1, wherein the artichoke comprises 110-250 mg, wherein the dandelion comprises 125-160 mg, wherein the milk thistle comprises 125-160 mg, wherein the alpha-lipoic acid comprises 125-160 mg, and wherein the turmeric comprises of 75-125 mg.

3. The method of claim 1, the amylase comprises 30 to 1,500 DU, wherein the glucoamylase comprises 0.055 to 3 AGU, wherein the protease 4.5 comprises 85 to 4,250 HUT, wherein the protease 6 comprises 10 to 500 HUT, wherein protease 3 comprises 0.43 to 22 SAPU, wherein the neutral protease comprises 16 to 800 PC, wherein the peptidase comprises 10 to 500 HUT, wherein the papain comprises 1.5 to 75 USP, wherein the alpha-galactosidase comprises 0.44 to 22 GAL, wherein the acid maltase comprises 0.045 to 2.5 U, wherein the cellulase comprises 75 to 3750 CU, wherein the pectinase comprises 0.03 to 15 HUT, wherein the protease inhibitor 6.0 comprises 4.55 to 230FIP, wherein the lipase comprises 3.5 to 15,250 ALU, wherein the lactase comprises 3.5 to 15,250 ALU, wherein the bromelain comprises 1.6 to 80 FCC PU, wherein the cellulase comprises 0.76 to 40 CU, wherein the xylanase comprises 1.08-50 XU, and wherein the invertase comprises 1.9 to 100 SU.

4. The method of claim 1, wherein the oral formulation may be administered up to three times daily for up to twelve weeks.

5. The method of claim 1, wherein the oral formulation may be administered as the herbal blend per dose of 35-1500 mg and the enzyme blend per dose as 25 mg to 200 mg.

6. The method of claim 1, wherein the isolated enzyme is isolated by at least one of the selected from the group consisting of mechanically isolated, chemically isolated, biochemically isolated, isolated by chromatography, solubilization, and any combination thereof.

7. The method of claim 1, wherein the isolated enzyme is sourced from a plant, recombinant protein via recombinant DNA/RNA technologies, an animal source, and any combination thereof.

8. A method of claim 1, wherein the liver dysfunction-related disease is selected from a group consisting of stalled weight loss, fatty liver, abnormal liver enzyme levels, weak and slow-growing hair and nail, dull and wrinkled skin, fatigue, type II diabetes, and hypercholesteremia.

9. A method of claim 1, wherein the liver dysfunction-related disease is gut dysfunction diseases selected from a group consisting of abnormal food digestion, abnormal mucosal blood flow, abnormal intestinal motility, abnormal gut lining permeability, abnormal nutrient absorption, enhanced allergen absorption, and ulcers.

10. A method of claim 1, wherein the liver dysfunction-related disease is abnormal kidney function.

11. A method of claim 1, wherein the liver dysfunction-related disease is an abnormal immune function, autoimmune diseases, and inflammation.

12. A method of claim 1, wherein the liver dysfunction-related disease is abnormal respiration.

13. A method of claim 1, wherein the liver dysfunction-related disease is infections.

14. A method of claim 1, wherein the liver dysfunction-related disease is alcohol abuse-related liver disease.

15. The method of claim 1, wherein the oral formulation increased physical energy by up to 100%.

16. The method of claim 1, wherein the oral formulation increased mental clarity by up to 30%.

17. The method of claim 1, wherein the oral formulation reduced the waist by up to 4 inches.

18. The method of claim 1, wherein the oral formulation reduced the weight by up to 40 pounds.

19. The method of claim 1, wherein the herbal blend is in an outer compartment and the enzyme blend is within an inner compartment.

20. The method of claim 19, wherein the inner compartment enzyme blend is resistant to gastric acid and released in the intestine.

21. The method of claim 19, wherein the outer compartment herbal bend is configured to release its content in the stomach, and wherein the stomach's acid may entirely or partially digest the herbal blend.

22. The method of claim 19, wherein the inner compartment is selected from the group consisting of hard-shelled capsules, soft-shelled capsules, drug-embedded capsules, entric coating, stomach acid-resistant capsules, Pullulan acid-resistant capsules, HPMC acid-resistant capsules, and delayed-release capsule.
